(19) United States
(12) Patent Application Publication
(10) Pub. No.: US 2005/0064037 A1
(43) Pub. Date: Mar. 24, 2005

(54) Transdermal delivery of oxybutynin in gel formulations

(76) Inventor: Chin-Chih Chiang, West Covina, CA (US)

Correspondence Address: SHIHONG NICOLAOU, 4931 ARROYO LINDO AVE., SAN DIEGO, CA, 92117, US

(21) Appl. No.: 10/770,088

(22) Filed: Feb. 2, 2004

(30) Foreign Application Priority Data
Sep. 18, 2003 (TW) 092125778

(51) Int. Cl. A61K 9/00; A61K 9/70; A61K 9/14

(52) U.S. Cl. 424/486; 424/449

(57) ABSTRACT

A topical gel formulation of oxybutynin is provided that exhibits enhanced bioavailability, minimized adverse side effects and skin irritations, and improved patient compliance compared to the existing oral and transdermal formulations of oxybutynin.

### RELATED APPLICATIONS

This application claims the benefit of priority of Taiwan Application Serial No. 092125778 filed Sep. 18, 2003, the disclosure of which is incorporated herein by reference in its entirety.

### FIELD OF THE INVENTION

The invention relates generally to the transdermal delivery of oxybutynin. More specifically, the invention provides the compositions and methods of use for gel formulations of oxybutynin therapeutic for topical administration, and the method of preparing the gel formulation and the products.

### BACKGROUND OF THE INVENTION

Oxybutynin is used for treating various forms of overactive bladder and urinary incontinence. Particularly, oxybutynin effectively treats neurogenically caused bladder disorders. Relief from such disorders is attributed to the anticholinergic and antispasmodic action which oxybutynin imparts to the parasympathetic nervous system and the urinary bladder detrusor muscle.

It is generally believed that, while this anticholinergic activity contributes to oxybutynin's clinical usefulness, it also contributes to certain uncomfortable adverse drug experiences such as dry mouth, dizziness, blurred vision, and constipation. More specifically, these experiences have been generally attributed to the presence and amount of active metabolites of oxybutynin, for example, N-desethyloxybutynin. The above-referenced adverse drug experiences are observed in a majority of patients using current oxybutynin formulations. In some cases, these adverse experiences are severe enough to persuade the patient to discontinue treatment.

Oral and transdermal oxybutynin administrations are currently used for treating various forms of overactive bladder and urinary incontinence. However, the bioavailability of the oral delivery is rather low, and the majority of the actives can not reach the systemic circulation. In addition, the adverse side effects caused by the active metabolites can be significant. The oral dosage forms are particularly inconvenient for the elders and the patients with swallowing difficulties.

Due to various disadvantages of oral dosage forms, transdermal adhesive matrix patches have been developed. For example, U.S. Pat. Nos. 6,555,129 and 6,562,368, and European Pat. No. 1174132A1 have demonstrated the transdermal therapies of oxybutynin. The transdermal delivery of oxybutynin can avoid the first-pass hepatic effect by directly introducing the drug into blood stream, and consequently enhance the bioavailability. The dose can be reduced and the adverse side effects can also be minimized by transdermal delivery of oxybutynin. However, the skin irritations caused by the transdermal adhesive matrix patches remain to be a problem. Sometimes, the irritation may discourage patients to discontinue the treatment, particularly for the long-term users.

Thus, the needs still remain for the improved formulations of oxybutynin, which may significantly reduce the adverse side effects and skin irritations.

### SUMMARY OF THE INVENTION

The present invention provides the compositions and methods of use for topical gel formulations of oxybutynin.

In one embodiment, the gel formulation comprises 0.5-5% (w/w) oxybutynin chloride salt, 10-80% (w/w) short chain alcohols, and 0.2-2.0% of gelling agents.

In another related aspect, the gel formulation comprises the permeation enhancers in order to increase the rate at which oxybutynin penetrates through the skin. Chemical enhancers are compounds that are administered along with the drug in order to increase the permeability of the stratum corneum, and thereby provide for enhanced penetration of the drug through the skin. Ideally, such chemical permeation enhancers are compounds that are innocuous and serve merely to facilitate diffusion of the drug through the stratum corneum.

The topical gel formulations of oxybutynin in the present invention have advantages including the following aspects.

1. Increased bioavailability of oxybutynin by direct drug absorption into bloodstream.

2. Reduced adverse side effects of oxybutynin.

3. Extended long-term effect, by once daily administration. Convenient and suitable for long term use.

4. Suitable for elders and patients with swallowing difficulties.

5. Minimized effect to liver, suitable to the patients with liver diseases.

6. Minimized drug interactions.

7. Reduced skin irritations than those of the adhesive matrix patches.

### BRIEF DESCRIPTION OF THE DRAWINGS

FIG. 1 illustrates the plasma concentrations of oxybutynin vs. time following the transdermal delivery of 1% topical gel formulation and 3% topical gel formulation.

### DETAILED DESCRIPTION OF THE INVENTION

The present invention provides the compositions of topical gel formulation of oxybutynin, which exhibits reduced adverse side effects and minimal skin irritation. Such topical gel formulation of oxybutynin is delivered by topical administration to systemic circulation.

In one embodiment, the gel formulation comprises 0.5-5% (w/w) oxybutynin chloride salt, 10-80% (w/w) short chain alcohols, and 0.2-2.0% of gelling agents. The preferred short chain alcohols are ethanol and isopropanol. The preferred gelling agents include Carbomer (a synthetic compound comprised of a cross-linked polymer of acrylic acid with a high molecular weight, including various products such as Carbopol ETD 2020 et al.) and Pemulen TR-1NF (a cross-linked copolymer of acrylic acid and C₁₀₋₃₀ alkyl acrylate).

In another embodiment, the gel formulation comprises the permeation enhancers in a range of 0.5-5.0% (w/w). The suitable permeation enhancers include propylene glycol, propylene glycol laurate, isopropyl myristate, and methyl lactate, and preferably with the use of isopropyl myristate. Additionally, moisturizers can be added in the formulation, such as propylene glycol.

The following examples of the gel formulations of oxybutynin are provided to further explain the invention.

### EXAMPLE 1

Preparations of Topical Gel Formulations of Oxybutynin

A gel formulation of oxybutynin was prepared by the following representative procedure.

1. Dilute propylene glycol in a water-containing vessel.

2. Slowly disperse Carbopol ETD 2020 in the propylene glycol/water solution as in step 1.

3. Mix propylene glycol with all other excipients as listed in Table 1, in a separate vessel.

4. Combine and mix the solutions in step 1 and step 2.

5. Dissolve oxybutynin chloride salt in the solution of step 4.

6. Adjust pH of the solution in step 5 to 6.5-7.5 using a base (such as 2-amine-2-methyl-1-isopropanol, or diisopropanolamine).

### EXAMPLE 2

Skin Permeation Rates of Various Gel Formulations

The skin permeation rates were measured for various gel formulations of oxybutynin as shown in Table 1.

TABLE 1
Compositions of the Gel Formulations of Oxybutynin and the Skin Permeation Rates

| | Formulation | Formulation | Formulation | Formulation | Formulation | Formulation | Formulation |
|---|---|---|---|---|---|---|---|
| | 1 | 2 | 3 | 4 | 5 | 6 | 7 |
| | (OXY004- | (OXY004- | (OXY004- | (OXY004- | (OXY004- | (OXY004- | (OXY004- |
| | 067a) | 067b) | 078c) | 078d) | 079g) | 079h) | 088a) |
| Compositions | % (w/w) | % (w/w) | % (w/w) | % (w/w) | % (w/w) | % (w/w) | % (w/w) |
| Purified Water | 40.0 | 34.0 | 38.0 | 40.0 | 39.0 | 39.0 | 40.0 |
| Carbopol ETD2020 | 1.0 | — | 1.0 | 1.0 | 1.0 | — | 1.0 |
| Pemulen TR-1NF | — | 1.0 | — | — | — | 1.0 | — |
| Propylene glycol | 2.0 | 2.0 | 4.0 | 2.0 | 4.0 | 2.0 | 2.0 |
| Isopropanol | 53.0 | 53.5 | 53.0 | 53.0 | 50.5 | 50.5 | 53.3 |
| Isopropyl myristate | 1.5 | — | 1.5 | — | 5.0 | 5.0 | — |
| Propylene glycol | — | 5.0 | — | — | — | — | 1.5 |
| laurate | | | | | | | |
| Methyl lactate | — | — | — | 1.5 | — | — | — |
| Lecithin* | — | 2.0 | — | — | — | — | — |
| Oxybutynin chloride | 1.0 | 1.0 | 1.0 | 1.0 | 1.0 | 1.0 | 1.0 |
| 2-amine-2-methyl-1- | 1.5 | 1.5 | 1.5 | 1.5 | 1.5 | 1.5 | 1.2 |
| isopropanol | | | | | | | |
| Skin permeation rate | 0.31 ± 0.09 | 0.11 ± 0.06 | 0.21 ± 0.03 | 0.16 ± 0.07 | 0.11 ± 0.02 | 0.10 ± 0.07 | 0.57 ± 0.06 |
| [mg/10 cm²/day, | (28.4%) | (53.5%) | (16.1%) | (41.1%) | (22.1%) | (65.8%) | (10.1%) |
| Mean ± STD (% RSD, | | | | | | | |
| n = 3)] | | | | | | | |

*Lecithin is used both as an emulsifying agent and as solubility enhancer.

### EXAMPLE 3

Pharmacokinetics of the Oxybutynin in Topical Gel Formulations

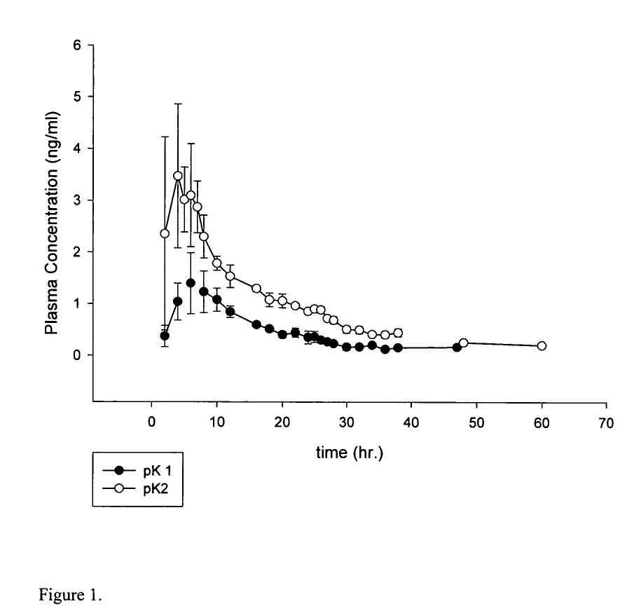

The pharmacokinetic studies were conducted by applying 5 gm of the 1% oxybutynin gel formulation to three male patients and 5 gm of 3% oxybutynin gel formulation to three female patients on the abdominal skin area. The compositions of 1% and 3% oxybutynin gel formulations are described in Table 2. FIG. 1 demonstrated the plasma concentrations of oxybutynin vs. time following the transdermal delivery of 1% topical gel formulation and 3% topical gel formulation.

TABLE 2
The compositions of 1% and 3% oxybutynin gel formulations used for the
pharmacokinetic studies

| | 1% Gel | 3% Gel |
|---|---|---|
| Composition | (w/w) | (w/w) |
| Purified water | 44.0 | 40.0 |
| Propylene glycol | 2.0 | 2.0 |
| Carbopol ETD2020 | 1.0 | 1.0 |
| Isopropanol | 50.0 | 50.0 |
| Propylene glycol laurate | 1.0 | 1.0 |
| Oxybutynin chloride | 1.0 | 3.0 |
| Diisopropanoamine | 1.0 | 3.0 |
| Sum | 100.0 | 100.0 |
| pH | 7.0-8.5 | 7.0-8.5 |

The comparison of the pharmacokinetic data of the single dose oral formulation of oxybutynin (Ditropan XL, 10 mg, patient number=43 *) and the gel formulations of oxybutynin of the present invention (patient number=3) is shown in Table 3. Cₘₐₓ represents the maximum drug concentration in the blood; while tₘₐₓ represents the time it takes to reach the Cₘₐₓ. t_{1/2} represents the half-life for the drug elimination. AUC₍₀₋₄₈₎ is the area under the curve of blood concentration vs. time from time 0 to 48 hours. AUC_{inf} is the area under the curve of the blood concentration from time 0 to infinity.

TABLE 3
Comparison of Pharmacokinetic (PK) Parameters of DITROPAN XL
(10 mg) and Oxybutynin Gel Formulations

| | | | | 1% Gel | 3% Gel |
|---|---|---|---|---|---|
| | DITROPAN XL 10 mg* | | | Formulation | Formulation |
| PK parameters | R-oxybutynin | S-oxybutynin | Sum | Oxybutynin | Oxybutynin |
| Cₘₐₓ (ng/mL) | 1.0 ± 0.6 | 1.8 ± 1.0 | 2.8 | 1.41 ± 0.59 | 61 3.47 ± 1.39 |
| tₘₐₓ (hour) | 12.7 ± 5.4 | 11.8 ± 5.3 | — | 6.67 ± 3.06 | 4.00 |
| t_{1/2} (hour) | 13.2 ± 6.2 | 12.4 ± 6.1 | — | — | 21.5 ± 0.8 |
| AUC₍₀₋₄₈₎ | 18.4 ± 10.3 | 34.2 ± 16.9 | 52.6 | 21.0 ± 2.7 | 54.7 ± 8.4 |
| (ng · hour/mL) | | | | | |
| AUC_{inf} | 21.3 ± 12.2 | 39.5 ± 21.2 | 60.8 | 33.6 ± 12.9 | 61.8 ± 9.9 |
| (ng · hour/mL) | | | | | |

*See Physicians' Desk Reference, p. 2453, 57 Edition, 2003.

As illustrated in Table 3, the blood concentration of oxybutynin is at a similar level in gel formulations of the present invention as in a conventional oral formulation. Since the topical gel formulation of oxybutynin in the present invention is delivered transdermally into blood stream, it avoids the first-pass hepatic effect. Consequently, the adverse side effects induced by the active metabolite of oxybutynin from an oral delivery can be minimized. The gel formulations also reduced the skin irritation comparing to the conventional adhesive matrix patch.

Although the invention has been described with reference to the examples described herein, it will be understood that modifications and variations are encompassed within the spirit and scope of the invention. Accordingly, the invention is limited only by the following claims.

1. A topical gel formulation of oxybutynin comprising:
0.5-5% (w/w) of oxybutynin chloride salt, 10-80% (w/w) of a short chain alcohol, and
0.2-2.0% (w/w) of a gelling agent.

2. The topical gel formulation as in claim 1, wherein the short chain alcohol is ethanol or isopropanol.

3. The topical gel formulation as in claim 1, wherein the gelling agent is Carbomer or Pemulen TR-1NF.

4. The topical gel formulation as in claim 3, wherein the Carbomer includes Carbopol ETD 2020.

5. The topical gel formulation as in claim 1, further comprises of 0.5-5.0% (w/w) permeation enhancer.

6. The topical gel formulation as in claim 5, wherein the permeation enhancer is selected from the group consisting of propylene glycol, propylene glycol laurate, isopropyl myristate, and methyl lactate.

7. The topical gel formulation as in claim 5, wherein the permeation enhancer is isopropyl myristate.

8. The topical gel formulation as in claim 1, further comprises of a moisturizer.

9. The topical gel formulation as in claim 8, wherein the moisturizer is propylene glycol.

10. A method for treating bladder disorders in a subject, said method comprising administrating to a subject in need thereof an effective amount of a topical gel formulation of oxybutynin according to claim 1.
